(19) United States
(12) Patent Application Publication
(10) Pub. No.: US 2021/0212320 A1
(43) Pub. Date: Jul. 15, 2021

(54) FRUIT THINNING METHOD WITH 1-AMINOCYCLOPROPANE CARBOXYLIC ACID

(71) Applicant: Valent BioSciences LLC, Libertyville, IL (US)

(72) Inventors: Franklin Paul Silverman, Highland Park, IL (US); Jim Hansen, Bensenville, IL (US); Gregory D. Venburg, Deerfield, IL (US); Derek D. Woolard, Zion, IL (US); Gregory G. Clarke, Dillsburg, PA (US); Michael Schroeder, Guggenhausen (DE); Andrew Rath, Underwood (AU); Peter D. Petracek, Grayslake, IL (US)

(21) Appl. No.: 17/216,897

(22) Filed: Mar. 30, 2021

(51) Int. Cl. A01N 37/44

(52) U.S. Cl. CPC A01N 37/44

(57) ABSTRACT

The present invention relates to fruit thinning method with 1-aminocyclopropane carboxylic acid (ACC) to reduce crop load of stone fruit trees or pome fruit trees.

### FIELD OF THE INVENTION

The present invention relates to fruit thinning method with 1-aminocyclopropane carboxylic acid (ACC) to reduce crop load of stone fruit trees or pome fruit trees.

### BACKGROUND OF THE INVENTION

Stone fruits such as almond, apricot, cherry, nectarine, peach, and plum are important perennial fruit crops in the US and around the world. There is an increasing emphasis on producing larger fruit of high quality, as opposed to volume of fruit (tonnage). Growers are now challenged to produce crops of uniformly large fruit with adequate color and optimal flavor as consumers have grown to expect high quality fruit on a year-round basis.

Reduction of the crop load on a tree (thinning) is often used to produce high quality tree fruit. During flowering and fruit set, growers commonly physically or chemically remove flowers (flower thinning) or young fruit (fruitlet thinning) to maximize the size and quality of the remaining fruit (Dennis, 2000, Plant Growth Reg. 31: 1-16). In general, the earlier the crop load is ‘thinned’ the better the quality of fruit at harvest. Removal of flowers or fruitlets on each tree by hand (hand thinning) often provides consistent results but can be prohibitively expensive. The use of chemicals for cost-effective flower or fruitlet thinning is preferable. The chemical insecticide carbaryl is often used for thinning apple fruitlets (Petracek et al., 2003, HortScience. 38: 937-942). However, carbaryl faces regulatory challenges and is no longer available to growers in some regions. The cytokinin 6-benzyladenine (6BA) is an important thinning chemical and is particularly effective for increasing fruit size. However, 6BA-induced thinning is sensitive to physiological and weather conditions (Yuan and Greene, 2000, J. Amer. Soc. Hort. Sci. 125: 169-176). For stone fruit such as peaches, there are currently no chemicals that safely and consistently induce post-bloom thinning (Costa and Vizzotto, 2000, Plant Growth Reg. 31: 113-119; Byers et al, 2003. In: Janick ed. Horticultural Reviews, John Wiley and Sons, Inc., 351-391). As Byers stated in 1978 (J. Amer. Soc. Hort. Sci. 103:232-236) “The search for an effective chemical peach thinning agent has not resulted in a commercially acceptable method of fruit removal. Numerous materials have been tried and most have been discarded due to inconsistent results, leaf abscission, fruit deformation, or unacceptable timing in relation to bloom and the frost period.” After more than 30 years since this publication, there is still a need for new chemicals that safely and consistently reduce crop load in these and other tree fruit crops.

ACC is paid attention as the new chemicals and many patent applications relating to ACC including for fruit thinning were published, including WO2010144779, WO2018183674, WO2018183680, WO20181836, WO2018207693, and WO2018207694. Each of these patent applications listed are incorporated by reference herein as the ACC salts, hydrates, polymorphs, and formulations disclosed in these patent applications may be used in methods of the present invention.

It is an object of the invention to reduce the crop load of pome fruits, and stone fruits, using appropriate amount of ACC or salts thereof, its formulations and application method during growing season.

### SUMMARY OF THE INVENTION

The present invention is directed to a method of thinning stone fruit and pome fruit by applying ACC as a foliar spray.

In this invention, ACC is not limited to its zwitterionic form. It may include naturally occurring metabolites such as alpha keto butyrate (Honma and Shimomura, Agric. Biol. Chem., 42, 1825. 1978), MalonylACC (MACC; Amrhein et al., Naturwissenschaften 68, 619. 1981) Gamma GlutamylACC (GACC; Martin et al., Plant Physiol. 109, 917. 1995) and the Jasmonic acid conjugate (JACC Staswick and Tiryaki, Plant Cell 16, 2117. 2004).

ACC can be used in the form of salt derived from inorganic or organic acids or bases. Acid addition salts of the active ingredients of the present invention can be prepared in situ during the final isolation and purification of the compounds of the invention or separately by reacting a free base function with a suitable organic acid. Representative acid addition salts include, but are not limited to acetate, adipate, alginate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form acid addition salts include such inorganic acids as hydrochloric acid, hydrobromic acid, hyaluronic acid, and phosphoric acid and such organic acids as oxalic acid, maleic acid, methanosulfonic acid, and succinic acid. Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

As the stone fruits, plum, peach, nectarine, cherry, apricot, almond and stonefruit hybrids thereof are exemplified. A person of skill in the art would understand that ACC would also be capable of promoting or causing thinning or increasing return bloom of future varieties, hybrids and cultivars of those fruits listed below.

As variety of plum, Sungold, Red Beauty, Golden Japan, Black Amber, Santa Rosa, Reina Claudia Verde, Reina Claudia de Oullins, Angelino, Formosa, Burbank, Stanley, Ana Spath, President, Giant, Baler Fruhzwetschge, Zimmers Fruhzwetschge, Hanka, Katinka, Ersinger, Elena, Presenta, Hanita, Hauszwetschge, Cacaks, President, Cacaks Beste, Ortenauer, Stanley, Valjevka, Herman, Fellenberg, Pitestean, Top and varieties, hybrids and cultivars thereof

As variety of peach, Autumnglo, Beekman, Blazingstar, Blushingstar, Bounty, Canadian Harmony, Contender, Coralstar, Country Sweet, Cresthaven, Crimson Lady, Crimson Rocket, Desiree, Early Loring, Earlystar, Encore, Early Red Fire, Early Rich, Ernies Choice, FlavrBurst, Gala Peach, Galaxy, Garnet Beauty, Glenglo, Glohaven, Gloria, Glowingstar, Harrow Beauty, Jerseyqueen, John Boy, John Boy II, Klondike White, Krista, Lady Nancy, Laurol, Loring, Madison, Manon, May Princess, Messina, NJF 18, NJF15, PF 1 Flamin Fury, PF 15A Flamin Fury, PF 17 Flamin Fury, PF 23 Flamin Fury, PF 25 Flamin Fury, PF 27A Flamin Fury, PF 28-007 Flamin Fury, PF 5B Flamin Fury, Raritan Rose, Redhaven, Redskin, Reliance, Rich Lady, Rich May, Salem, Saturn, Sentry, Snowbrite, Spring Flame series, Spring Snow, Starfire, Sugar Giant, Sugar May, SummerFest, Suncrest, Sunhigh, Sweet Breeze, Sweet Dream, Sweet Scarlet, Sweet-N-Up, Tangos, Tangos II, Victoria, White Lady Zee Diamond, Maria Blanca, Large White, Iris Roso, Flordalgo, Maria Delicia, Alexandra, Springcrest, Spring Lady, SpringBelle, St. Isidoro, Royal Glory, Rich Lady, Redtop, Maria Rosa, Maycrest, Early Maycrest, Flavorcrest, Early grande, Queen Crest, Starcrest, An-dross, Catherina, Everts, Suney, Tirrenia, Ionia, Maria Serena, Federica, Romea, Carson, Muntaingold, Babygold, Sudanell, Fantasia and Suncrest and varieties, hybrids and cultivars thereof.

As variety of nectarine, Armredark, August Red, Crimson Baby, Early Red 1, Early Red 2, Fairlaine, Fantasia, Firebright, Late Fantasia, Mary Emilia, Mayfire, Mayglo, Maygrand, Maylis, NB1420, NB2024, Nectaross, Orion, Queen Giant, Redfree, Redgold, Rose Diamond, Silverking, Snow Queen, Springred, Sunglo, Tasty Gold, Venus, YFN 13/02, NB-1524, Monnail, Caldesi 2000, Syller, Big-Top, Majestic Giant, Silver Gem, G. Diamond, Flavor Giant, Saphir, Alesandra, Mid Silver, Monnaze, Silver Late, Armking, Primerinque, Pacific Star, Sunfree, NJN-76, Maria Laura, Flavorgolg, Stark Sunglo, Antares, Magali, Nataly, Stark Redgold, Maria Aurelia, Sweet Red, Seleccion 1989 and Harvest Sun and varieties, hybrids and cultivars thereof.

As variety of cherry, Balck Tartarian, Early Burlat, Mona, Berryessa, Brooks, Chelan, Bada, Chinook, Coral, Corum, Larian, Tieton, Tulare, Index, Garnet, Ruby, Julbilee, King, Cristalina, Benton, Bing, Sweet Ann, Van, Summit, Rainier, Utah Giant, Stella, Lambert, Selah, Attika, Lapins, Skenna, Sweetheat, Craig, Star Crimson, Attika, Mona, Rainier, Skenna, Tieton, Tulare, Napoleon, Ambrunesa, Sunburst, Duroni 3, Early Van Compact, Hedelfinger, Schneiders, Burlat, Meckenheimer, Kordia, Regina, Vanda, Vic, Viola, Valeska, Staccato, Rubin, Sam, Oktavia and Alma and varieties, hybrids and cultivars thereof.

As variety of almond, Sonora, Winters, Avalon, Durango, Aldrich, Price, Nonpareil, Wood Colony, Wood Colony, Carmel, Monterey, Butte, Padre, Mission, and Ruby, Antolieta, Ayles, Belona, Blanquerna, Cambra, Felisia, Mardia, Marta, Penta, Soleta, Tardona, Vialfas, Marcona, Desmayo Largueta, Ferragnes, Guara, Masbovera, Glorieta, Francoli, Vayro, Marinada, Constanti and Tarraco and varieties, hybrids and cultivars thereof.

As the pome fruits, apple and pear, Asian pear and Japanese pear are exemplified. A person of skill in the art would understand that ACC would also be capable of promoting or causing thinning our increasing return bloom of future varieties, hybrids and cultivars of those fruits listed below.

As variety of apple, Acey Mac, Acey Mac, Albemarle Pippin, Arctic™ Apple, Arkansas Black, Arlet/Swiss Gourmet, Ashmead's Kernel, Autumn Crisp, Autumn Gala, Baldwin, Beacon, Blondee, Braeburn, Bramley's Seedling, Brookfield Gala, Buckeye Gala, Burgundy, Calville Blanc,

Cameo, Chrisolyn Jonathan, Cornish Gilliflower, Cortland, Court Pendu Plat, Cox's Orange Pippin, Crimson Gala, Crimson Gold, Crimson Topaz, CrimsonCrisp, Cripp's Pink, Crown, Empire, Dandee Red, Daybreak Fuji, Duchess of Oldenb, urg, Egremont Russet, Ellison's Orange, Elstar, Empire, Enterprise, Florina, Freedom, Freyberg, Fuji (Brak Cltv), Fulford Gala, Galarina, Gale Gala, Ginger, Gold, Golden Delicious, Golden Russet, GoldRush, Granny Smith, Gravenstein, Grimes Golden, Hampshire Mac, Hardy Cumberland, Honeycrisp, Idared, Initial, James Grieve, Jonagold De Coster, Jonamac, Keepsake, Kidd's Orange Red, King of Tompkins County, Kumeu Crimson Braeburn, Lady, Laxton's Superb, Liberty, LindaMac, Lodi, Macoun, Marshall McIntosh, McIntosh, Melrose, Mollies Delicious, Mutsu, Newtown Pippin, Nittany, Northern Spy, Nova Spy, Orleans Reinette, Pink Lady brand Cripps Pink Variety, Pristine, Querina, Red Jonaprince, Red Rome Beauty, Red Winesap, Red Yorking, Redfree, Rhode Island Greening, Ribston Pippin, Rogers Red McIntosh, Roxbury Russet, Royal Court, Royal Empire, RubyMac, Saint Edmund's Russet, Sansa, Shizuka, Smokehouse, Snapp Stayman, SnowSweet, Spartan, Spitzenburg, Spur Winter Banana, Sturmer Pippin, Summer Rambo, SunCrisp, Super Chief Spur Red Delicious, Sweet Sixteen, Tydeman's Late Orange, Ultima Gala, WineCrisp, Wolf River, Worcester Pearmain, Yellow Transparent, Zabergau Reinette, Zestar, Starking, Richared, Starkrimson, Reineta blanca del Canada, Verde doncella, Galaxy, Roma beauty, Kanzi, Topaz, Alkmene, Rewena, Pinova, Pilot, Boskoop, Shampion, Ligol, Gloster, Caudle, Tsugaru, Stayman, Hokuto, Lobo and Jazz and varieties, hybrids and cultivars thereof.

As variety of pear, Asian pear and Japanese pear, Anjou, Red Anjou, Asian Pears, Bartletts, Red Sensation Bartlett, Reimer Red, Bosc, Cascade, Comice, Concorde, Conference, Forelle, French Butter, Starkrimson, Seckel, First pear, New Juice, Juice of Good Fortune, New Century, Abundant Juice, Plentiful, Sapphire, 20th Century, Good Pear, New Success, Daisui Li, Shin Li, Olympic, Floating Chrysanthemum, Duck Pear, New Quantity, Sweet Pear, Atago, Seuri, Madame Luck, Sweet ‘N’ Sour, Sunburst, Autumn Sweet Williams Christ, Limonera, Bonne Louise, Blanquilla, Conference, Abate Fetel, Passacrassana, Alexander Lucas, Hardy, Packham's Triumph, Xenia, Santa Maria, Dessertnaja, Harrow Delight, Gute Luise, Kaiser Alexander, Vienne, Nashi, Tongern, Condo, Tristan, Uta, Dicolor, Noj abrskaj a, Hortensia, Isolda, Herrmann, Gute Luise, Clapps Liebling, Gellerts Butterbirne and Bosc Flaschenbirne and varieties, hybrids and cultivars thereof.

The plant of the variety mentioned above may be a plant which can be produced by natural hybridization, a plant which can occur as the result of a mutation, an F1 hybrid plant, or a transgenic plant (also referred to as a “genetically modified plant”). These plants generally have properties such as a property that the tolerance to an herbicide is imparted, a property that a toxic substance against pests is accumulated, a property that the sensitivity to a plant disease is suppressed, a property that yield potential is increased, a property that the resistance to a biological or non-biological stress factor is improved, a property that a substance is accumulated, and improvement in a storage property or processability.

The term “F1 hybrid plant” refers to a plant of a first filial generation which is produced by hybridizing two different varieties with each other and is generally a plant which has a more superior trait to that of either one of parents thereof, i.e., has a hybrid vigor property. The term “transgenic plant” refers to a plant which is produced by introducing a foreign gene from another organism such as a microorganism into a plant and which has a property that cannot be acquired easily by hybridization breeding, induction of a mutation or a naturally occurring recombination under a natural environment.

Examples of the technique for producing the above-mentioned plants include a conventional breeding technique, a transgenic technique, a genome-based breeding technique, a new breeding technique, and a genome editing technique. The conventional breeding technique is a technique for producing a plant having a desirable property by mutation or hybridization. The transgenic technique is a technique for imparting a new property to a specific organism (e.g., a microorganism) by isolating a gene (DNA) of interest from the organism and then introducing the gene (DNA) into the genome of another target organism, and an antisense technique or an RNA interference technique which is a technique for imparting a new or improved property to a plant by silencing another gene occurring in the plant. As an example, Arctic™ Apples have been genetically modified to reduce the expression of polyphenol oxidase.

The genome-based breeding technique is a technique for increasing the efficiency of breeding using genomic information and includes a DNA marker (also referred to as “genome marker” or “gene marker”) breeding technique and genomic selection. For example, the DNA marker breeding is a method in which an offspring having a desired useful trait gene is selected from many hybrid offspring using a DNA marker that is a DNA sequence capable of serving as an indicator of the position of a specific useful trait gene on a genome. The analysis of a hybrid offspring of a plant at a seedling stage thereof using the DNA marker has such a characteristic that it becomes possible to shorten the time required for breeding effectively.

The genomic selection is such a technique that a prediction equation is produced from a phenotype and genomic information both obtained in advance and then a property is predicted from the prediction equation and the genomic information without carrying out the evaluation of the phenotype. The genomic selection can contribute to the increase in efficiency of breeding. A “new breeding technique” is a collective term for a variety of breeding techniques including molecular biological techniques. Examples of the new breeding technique include techniques such as cisgenesis/intragenesis, oligonucleotide-directed mutagenesis, RNA-dependent DNA methylation, genome editing, grafting to a GM rootstock or scion, reverse breeding, agroinfiltration, and seed production technology (SPT). The genome editing technique is a technique that converts genetic information in a sequence-specific manner, and enables addition, deletion and or substitution of a DNA base-pair sequence, addition, deletion and or substitution of an amino acid sequence, introduction of a foreign DNA base-pair sequence including genes and regulatory regions, and the like. Examples of the tool for the technique include zinc-finger nuclease (ZFN), TALEN, CRISPR/Cas9, CRISPER/Cpfl and meganuclease which can cleave DNA in a sequence-specific manner, and a sequence-specific genome modification technique using CAS9 nickase, Target-AID and the like which is produced by any one of the modification of the above-mentioned tools. A skilled artisan would understand that future techniques will be developed that are capable of editing the genomic sequence, modifying transcription of a DNA sequence to an RNA sequence, modifying an RNA sequence, modifying translation of an RNA sequence to an amino acid sequence, modifying an amino acid sequence and or modifying the folding of an amino acid sequence and or agglomeration of amino acid sequences to a protein and that any or all of these techniques may be beneficial in modifying the phenotype of a plant. Plants whose phenotypes have been modified by all known and future techniques capable of modifying the phenotype of a plant are envisaged herein.

Examples of the above-mentioned plants include plants listed in genetically modified crops registration database (GM APPROVAL DATABASE) in an electric information site in INTERNATIONAL SERVICE for the ACQUISITION of AGRI-BIOTECH APPLICATIONS, ISAAA) (http://www.isaaa.org/). More specific examples of the plans include an herbicide-tolerant plant, a pest-resistant plant, a plant disease-resistant plant, a plant of which the quality (e.g., the increase or decrease in content or the change in composition) of a product (e.g., starch, amino acid, fatty acid, etc.) is modified, a fertility trait modified plant, a non-biological stress-tolerant plant or a plant of which a trait associated with growth or yield is modified.

The present composition is usually a formulation prepared by mixing ACC with a carrier such as a solid carrier and a liquid carrier and adding adjuvants for formulation such as surfactants as necessary and dispersants. The formulation type is preferably an oil dispersion, a wettable powder, a water dispersible granule, a granule, a soluble solution, a dispersible concentrate, a suspension concentrate, and an emulsifiable concentrate with/or without encapsulation and/or controlled release.

In the present invention, ACC may be tank-mixed with an adjuvant. included, but not limited to, stickers, penetrators, UV stabilizer, acidifiers, water conditioners and the like.

Usually, ACC is mixed with an inert carrier, and if necessary, adding a surfactant or other auxiliaries for formulation, and then formulated as a water-dispersible granule, a water-soluble granule, a wettable powder, a water-soluble powder, soluble concentrate and the others.

Suitable inert carriers used upon formulation include solid carriers and liquid carriers including surfactants.

Examples of the other auxiliaries for formulation include binders, thickeners, preservatives, anti-freezing agents, and anti-foaming agents.

Throughout the application, all disclosed numerical ranges include all possible points (e.g., integers and decimals) within those ranges. All possible points within the ranges disclosed in the application can also be used as endpoints for ranges between these points. For example, a range of 0.01 to 99.99% includes 0.02% . . . 0.021% . . . 0.03% . . . 1% . . . 99.98% etc. and all ranges made up of these integers and decimals.

The total content of ACC in the present composition is usually within a range of 0.01 to 99.99% by weight, preferably 1 to 80% by weight, and more preferably 5 to 50% by weight, 0.01, 0.02, 0.03, 0.04, 0.05, 0.06. 0.07, 0.08, 0.09, 0.10, 0.125, 0.150, 0.175, 0.2, 0.3, 0.4, 0.5, 0.6, 0.7, 0.8, 0.9, 1, 5, 10, 20, 30, 40, 50, 60, 70, 80, 90 and 99.99% by weight and any more specific amount therein.

ACC and its formulations may be applied to plant foliage, fruit, flowers, seeds, bark, dormant and non-dormant buds, propagules, directly to the vasculature, roots or to the root zone via numerous application technologies. These technologies include, but are not limited to spraying, dipping, painting, injecting, seed treatment, painting, rubbing, in furrow treatments and soil injection. ACC or its formulations may be applied one or more times in a growing season including but not limited to a range of one to 1,000 times, one to twenty times and one, two, three, four, five, six, seven, eight, nine, ten, eleven, twelve, thirteen, fourteen, fifteen, sixteen, seventeen, eighteen, nineteen and twenty times.

Spraying the present composition may be performed by spraying a spray liquid obtained by mixing the ACC or its salts with water, using a spraying machine. In this case, concentration of ACC contains may be 0.0005 to 2% by weight, and preferably 0.005 to 1% by weight, 0.005, 0.006, 0.007, 0.008, 0.009, 0.010, 0.011, 0.012, 0.013, 0.014, 0.015, 0.016, 0.017, 0.018, 0.019, 0.020, 0.025, 0.030, 0.035, 0.040, 0.045, 0.050, 0.060, 0.070, 0.080, 0.090, 0.1, 0.2, 0.3, 0.4, 0.5, 0.6, 0.7, 0.8, 0.9, 1 and 2% by weight and any more specific amounts therein.

The spray liquid amount is not particularly limited and may be 1 to 10,000 L/ha, preferably 100 to 2000 L/ha, and more preferably 150 to 1000 L/ha, 1, 2, 3, 4, 5, 6, 7, 8, 9, 10, 20, 30, 40, 50, 60, 70, 80, 90, 100, 125, 150, 175, 200, 300, 400, 500, 600, 700, 800, 900, 1000, 2000, 3000, 4000, 5000, 6000, 7000, 800, 9000 and 10,000 L/ha and any more specific amounts therein.

Each application rate of ACC in the method of the present invention may be varied depending on a kind of plant to be applied, a formulation type, an application period, an application method, an application site, a climate condition, and the like. A total amount of ACC is within the range of usually 1.0 to 5000 g per hectare, preferably 1 to 2000 g per hectare, and further preferably 50 to 1500 g, per ha, 1, 2, 3, 4, 5, 6, 7, 8, 9, 10, 20, 30, 40, 50, 60, 70, 80, 90, 100, 125, 150, 175, 200, 300, 400, 500, 600, 700, 800, 900, 1000, 1500, 2000, 3000, 4000, and 5000 g per hectare and any more specific amounts therein.

Each application timing of ACC in the method of the present invention may be also varied depending on a kind of plant to be applied, a formulation type, an application period, an application method, an application site, a climate condition, and the like. With respect to application timing, application at bloom, post bloom, shuck split, late shuck split petal fall, 10 mm fruit stage, 25 mm, 35 mm fruit stage are exemplified.

The above representative embodiments are in no way limiting and are described solely to illustrate some aspects of the invention.

Further, the following examples are offered by way of illustration only and not by way of limitation.

### EXAMPLES

### Example 1

Binder solution is prepared by mixing binder(s), surfactant(s) and water. The binder solution is sprayed onto an ACC active ingredient and inert carrier dry powder mixture to form a wet mass. The wet mass is fed to an extruder. The extruder forces the wet mass through a die having 1.0 mm pores forming an extrudate. The extrudate is then dried resulting in a water-soluble granule comprising ACC. Composition of the water-soluble granule can be seen in Table 1, below.

TABLE 1

| | Formulation | Formulation | Formulation |
|---|---|---|---|
| Composition | Example 1 | Example 2 | Example 3 |
| ACC free acid | 20% | 30% | 40% |
| Silwet ® ECO spreader | 0.1% | 0.1% | 0.1% |
| Polysorbate 20 | 2.5% | 2.5% | 2.5% |
| (Tween ® 20) | | | |
| Polyvinylpyrrolidone | 1.1% | 1.1% | 1.0% |
| Lactose | to 100% | to 100% | to 100% |

% based on weight by total weight of the composition

Inert carrier-Lactose.

Surfactant-Silwet® ECO spreader; polyalkyleneoxide modified heptamethyltrisiloxane, Polysorbate 20, polyoxyethylene sorbitan monolaurate.

Binder-Polyvinylpyrrolidone.

### Examples 2 and 3

Water-soluble granules comprising ACC are prepared by the process of Example 1, above. Composition of the water-soluble granules can be seen in Table 1, above.

### Example 4

A water-soluble granule comprising ACC is prepared by the process of Example 1, above. Composition of the water-soluble granule can be seen in Table 2, below.

TABLE 2

| Composition | Example 4 | Example 5 | Example 6 | Example 7 |
|---|---|---|---|---|
| ACC free acid | 40% | 40% | 40% | 40% |
| Silwet ® ECO spreader | 0.1% | 0.1% | — | 0.1% |
| Break-Thru ® 200 | — | — | 0.1% | — |
| Polysorbate 20 | 2.5% | — | 2.0% | — |
| (Tween ® 20) | | | | |
| Polyvinylpyrrolidone | 1.0% | 1.0% | 1.0% | 1.0% |
| Brij ® 020 | — | 2.5% | — | — |
| Sodium Lignosulfonate | — | — | 1.0% | — |
| Aerosol ® OT-B | — | — | — | 2.5% |
| Lactose | to 100% | to 100% | to 100% | to 100% |

% based on weight by total weight of the composition

Inert carrier-Lactose.

Surfactant-Silwet® ECO spreader; polyalkyleneoxide modified heptamethyltrisiloxane, Break-Thru®; Polyether trisiloxane, Polysorbate 20; polyoxyethylene sorbitan monolaurate, Brij® 020; Polyoxyethylene vegetable-based fatty ether derived from cetyl alcohol, Aerosol® OT-B; Dioctyl sulfosuccinate sodium salt.

Binder-Polyvinylpyrrolidone.

### Examples 5 to 7

Water-soluble granules comprising ACC are prepared by the process of Example 1, above. Composition of the water-soluble granules can be seen in Table 2, above.

### Example 8

A water-soluble granule comprising ACC is prepared by the process of Example 1, above. Composition of the water-soluble granule can be seen in Table 3, below.

TABLE 3

| Composition | Example 8 | Example 9 | Example10 | Example 11 |
|---|---|---|---|---|
| ACC free acid | 40% | 40% | 40% | 40% |
| Polyvinylpyrrolidone | 0.8% | 0.8% | 0.8% | 0.8% |
| Polysorbate 20 | 1.5% | 1.5% | 1.5% | 1.5% |
| (Tween ® 20) | | | | |
| Calcium chloride | — | 5% | 10% | 20% |
| Lactose monohydrate | to 100% | to 100% | to 100% | to 100% |

% based on weight by total weight of the composition

Inert carrier-Lactose monohydrate, Calcium chloride.

Surfactant-Polysorbate 20; polyoxyethylene sorbitan monolaurate.

Binder-Polyvinylpyrrolidone.

### Examples 9 to 11

Water-soluble granules comprising ACC are prepared by the process of Example 1, above. Composition of the water-soluble granules can be seen in Table 3, above.

### Example 12

A water-soluble granule comprising ACC is prepared by the process of Example 1, above. Composition of the water-soluble granule can be seen in Table 4, below.

TABLE 4

| | Example | Example | Example | Example |
|---|---|---|---|---|
| Composition | 12 | 13 | 14 | 15 |
| ACC free acid | 40.0% | 40.0% | 40.0% | 40.0% |
| Break-Thru ® 240 | 0.1% | 0.1% | 0.1% | 0.25% |
| Polysorbate 20 | 2.5% | 2.5% | 2.5% | 2.5% |
| (Tweet ® 20) | | | | |
| Polyvinylpyrrolidone | 1.0% | 1.0% | 1.0% | 1.0% |
| Aerosol ® OT-B | 1.0% | — | — | — |
| Citric acid | 1.75% | 1.0% | 0.5% | 0.25% |
| Lactose | to 100% | to 100% | to 100% | to 100% |

% based on weight by total weight of the composition

Inert carrier-Lactose.

Surfactant-Break-Thru®; Polyether trisiloxane, Polysorbate 20; polyoxyethylene sorbitan monolaurate, Aerosol® OT-B; Dioctyl sulfosuccinate sodium salt.

Binder-Polyvinylpyrrolidone. pH adjuster-Citric acid.

### Examples 13 to 15

Water-soluble granules comprising ACC are prepared by the process of Example 1, above. Composition of the water-soluble granules can be seen in Table 4, above.

### Example 16

Aqueous stable agricultural formulations comprising ACC, water and calcium chloride, wherein the molar ratio of ACC to calcium chloride is from about 1.59:1 to about 1:2.27.

In this formulation example, ACC is present at a concentration from about 5% to about 40% w/w or from about 5% to about 25% w/w or from 5% to about 15% w/w or from about 10% w/w to about 25% w/w. In this example, the calcium chloride is present at a concentration from about 3.5% to about 75% w/w or from about 3.5% to about 35% w/w, or from about 7% to about 75% w/w. Moreover, this formulation further comprise a chelating agent, preferably, ethylenediaminetetraacetic acid (“EDTA”), preferably at a concentration from about 0.1% to about 0.2% w/w.

The formulation of this example provides very stable aqueous formulations for foliar spray, drench, in-furrow and seed treatment applications.

### Examples 17 and 18

In this example a liquid agricultural formulation comprising ACC HCl salt and either water or anon-aqueous solvent, wherein the formulation has a pH from 2.75.+−0.0.3 to 8.5.+−0.0.3. In the formulation, the ACC HCl salt is at a concentration from about 1% to about 50% w/w, preferably from about 5% to about 20% w/w. The formulations comprise a non-ionic surfactant.

In the aqueous example formulation comprising: about 5% to about 20% w/w ACC HCl salt, preferably about 13.6% w/w; about 1% to about 5% w/w polyoxyethylene alkyl ether phosphate with a degree of ethoxylation of 5 to 6 moles, preferably about 2.5% w/w; about 0.1% to about 1.5% w/w EDTA, preferably about 0.5% w/w; and water, wherein the formulation has a pH from 2.75.+−0.0.3 to 8.5.+−0.0.3, preferably from 4.0.+−0.0.3 to 8.0.+−0.0.3.

In non-aqueous agricultural formulation comprising: about 5% to about 20% w/w ACC HCl salt, preferably about 13.6% w/w; about 60% to about 90% propylene glycol, preferably about 84% w/w; and about 1% to about 5% w/w polysorbate 20, preferably about 2.0% w/w. Other possible solvents include, but are not limited to: propylene glycol and polyethylene glycol, dipropylene glycol, polypropylene glycol and butyl glycol.

The formulations of this example provide very stable aqueous and non-aqueous formulations for foliar spray, drench, in-furrow and seed treatment applications.

### Use of ACC for Fruit Thinning

In the following examples, the uses of ACC or its formulations for modulating fruit set are presented. These examples include but are not limiting to the rates presented.

### Example 19

### Applications to Stonefruit or Stonefruit Hybrids

Applications to stonefruit include, but are not limited to Apricot, Sweet and Tart Cherry, Nectarine, Peach, Plum, Chickasaw plum, Damson Plum, Japanese plum, Plumcot, Fresh Prune. Depending on cultivar, orchard conditions, application timing, and grower objectives, one or more of the following benefits will be associated with ACC are fruit thinning and/or enhanced return bloom

Use directions: Apply 300 to 600 ppm of ACC using sufficient spray volume to ensure complete tree coverage.

Note: Direct 80% of the spray into the upper ⅔rd of the tree canopy. Use higher rates in orchards that have a history of being difficult to thin and in varieties known to be difficult to thin. Do not apply ACC if temperatures are expected to fall below 32 F or exceed 90 F on the day of application.

### Example 20

### Applications to Pome Fruit

Depending on cultivar, orchard conditions, application timing, and grower objectives, one or more of the following benefits will be associated with ACC: Fruit thinning or Enhanced return bloom.

Use directions: Apply 200 to 400 ppm of ACC or its formulations using sufficient spray volume to ensure complete tree coverage. ACC can be applied in the period from full bloom until the average diameter of the king fruitlets is 25 mm. ACC is most active when king fruitlet diameter is 15-20 mm.

Note: Direct 80% of the spray into the upper ⅔rd of the tree canopy. Use higher rates in orchards that have a history of being difficult to thin, in varieties known to be difficult to thin, and in cool weather situations. Use ACC in a program with other thinning products, but do not apply ACC as a tank mix partner with other thinning products. Consider reducing the rate of application if temperatures are expected to exceed 90° F. on the day of application. Allow 7-10 days to observe the effect of any thinning product before making another application.

### Example 21

A spray liquid is obtained by mixing any one of the formulations of Examples 1-15, above, with water so that the concentration of ACC is 100 ppm. The spray liquid is sprayed on Babygold #5 peach trees at bloom as a target fruit. In that case the spray volume is 1000 L/ha. An acceptable chemical thinning agent is one that gives a substantial and relatively consistent reduction in crop load by the dose of ACC.

### Examples 22-27

The application is performed in the same manner as example 19, except that a target fruit, the concentration of ACC in the spray liquid, application volume of the spray liquid, and/or application timing are changed as shown in Table 5, below.

An acceptable chemical thinning agent is one that gives a substantial and relatively consistent reduction in crop load by each dose of ACC.

TABLE 5

| | Concentration | Application | | |
|---|---|---|---|---|
| Example | of ACC in | volume of the | Target | Application |
| name | spray liquid | spray liquid | fruit | timing |
| Test | 100 ppm | 1000 L/ha | Babygold #5 | bloom |
| Example 4 | | | | |
| Test | 100 ppm | 1000 L/ha | Babygold #5 | shuck split |
| Example 5 | | | | |
| Test | 100 ppm | 1000 L/ha | Babygold #5 | late shuck fall |
| Example 6 | | | | |
| Test | 100 ppm | 1000 L/ha | Babygold #5 | petal fall |
| Example 7 | | | | |
| Test | 100 ppm | 1000 L/ha | Babygold #5 | l0 nm fruit |
| Example 8 | | | | stage |
| Test | 100 ppm | 1000 L/ha | Babygold #5 | 20 nm fruit |
| Example 9 | | | | stage |

### Example 28

Effect of ACC concentration on fruit set (number of fruit per shoot) of Sugar May peaches and Sweet Dream peaches when applied at full bloom

TABLE 6

| ACC Concentration | Number of Fruit Per Shoot | |
|---|---|---|
| (parts per million) | Sugar May | Sweet Dream |
| 0 | 3.3 | 16 |
| 300 | 0.7 | 12 |
| 600 | 0.3 | 9 |
| 900 | 0.1 | 6 |

### Example 29

Effect of ACC concentration on modulating fruit set (number of fruit per 100 flower clusters) of Gala apples when applied at petal fall (BBCH67), 10 mm fruit diameter, or 20 mm fruit diameter.

TABLE 7

| | Time of Application | | |
|---|---|---|---|
| ACC Concentration | Petal | 10 mm fruit | 20 mm fruit |
| (parts per million) | Fall | diameter | diameter |
| 0 | 130 | | |
| 224 | 117 | 125 | 89 |
| 450 | 96 | 108 | 46 |
| 900 | 72 | 63 | 12 |

What is claimed is:

1. A method to reduce crop load comprising applying an effective amount of 1-aminocyclopropane carboxylic acid, a hydrate thereof, a polymorph thereof or a salt thereof to stone fruit trees or pome fruit trees.
